(19) United States
(12) Patent Application Publication
(10) Pub. No.: US 2008/0015414 A1
(43) Pub. Date: Jan. 17, 2008

(54) Combined Dispenser and Mouthpiece for Endoscope

(75) Inventors: Yakov BAR OR, Haifa (IL); Adi STRAUSS, Yokneam Moshava (IL)

Correspondence Address: BRUCE E. LILLING;LILLING & LILLING PLLC, P.O. BOX 560, GOLDEN BRIDGE, NY, 10526, US

(73) Assignee: STRYKER GI LTD., Haifa (IL)

(21) Appl. No.: 11/776,645

(22) Filed: Jul. 12, 2007

Related U.S. Application Data
(60) Provisional application No. 60/807,281, filed on Jul. 13, 2006.

(51) Int. Cl. A61B 1/00

(52) U.S. Cl. 600/114; 600/121; 600/124

(57) ABSTRACT

A combined component for use with an endoscopic apparatus for inspecting a body channel of gastrointestinal system of a patient is disclosed. The endoscopic apparatus comprises an insertion tube coverable by a covering sleeve deployable on the insertion tube upon advancing the insertion tube into the body channel. The combined component comprises an outer housing portion and an inner bushing portion. The inner bushing portion is receivable within the housing portion such that an annular space is provided therebetween for retaining the covering sleeve thereinto. The outer housing portion is provided with a bite portion, to be grasped between the patient's teeth.

### FIELD OF THE INVENTION

The present invention relates generally to the field of endoscopy and specifically to flexible endoscopes used for medical examination of gastrointestinal system during which an insertion tube provided with an optical head is advanced through the mouth down the esophagus. An example of possible implementation of the present invention could be a gastroscope for viewing the stomach or duodenum and for removing tissues from these organs. It should be kept in mind, however that the present invention is not limited to gastroscopes and can be implemented in any other endoscopes employed for medical examination of the gastrointestinal system, e.g. in duodenoscopes, sigmoidoscopes, etc.

### BACKGROUND OF THE INVENTION

The main components of a modern gastroscopic apparatus comprise a flexible insertion tube fitted at its distal end with an optical head, an operating handle for manipulating the insertion tube during its advancement within the body organ and a system control unit provided with a source of compressed air, water and vacuum to be supplied to the body organ during the gastroscopic procedure. The insertion tube is introduced within a patient's mouth through a dedicated mouthpiece held by the patient's teeth. The mouthpiece guides the insertion tube during the gastroscopic procedure.

Flexible endoscopes in general and gastroscopes in particular are notoriously difficult to clean and disinfect thoroughly, leading to problems of cross-contamination between patients and between patients and staff. These problems can be partially avoided by covering the endoscope with a single-use sleeve, which is discarded after use. The use of a disposable sleeve (also referred to as a sheath) to cover insertion tube of an endoscope is well known in the art.

Endoscopes commonly have working channels, running from a proximal port outside the body to a distal port at the distal end of the endoscope. When the distal end of the endoscope is inserted into the body organ, the working channel may be used to pass a surgical instrument through to the distal end of the endoscope in order to perform a surgical procedure, such as a biopsy.

Instruments that are used in this manner become contaminated with biological matter from inside the patient's body. When contaminated instrument is withdrawn from the body it spreads the contamination to the interior of the working channel and to the proximal port of the endoscope and eventually to the operator's hands.

Therefore it would be desirable to prevent spread of contamination originating from the endoscope itself or from the surgical instrument.

Ouchi (US Publication 2003/0097043) describes a cover for preventing contamination of an operating portion of an endoscope. The cover is formed in a bag-like shape for enveloping a total of the operating portion. The cover can prevent exposure to contamination during the endoscopic procedure when the insertion tube is inside the patient. However, during the procedure and in the end of the procedure, the insertion tube is withdrawn from the patient and, since it remains uncovered, the spread of contamination originating from the insertion tube would not be prevented.

Chinese patent CN 1,486,666 describes an endoscope system fitted with a disposable sheath, which at least at its distal end is made of transparent material. The sheath seals all the inserted portion of the endoscope. Various hard-to-clean, open-ended channels of the endoscope, including working channel for surgical instrument, are arranged outside the insertion tube and are disposed of together with the sheath after single use.

Still further methods for sheathing an endoscope for protecting it from contamination are described, for example, in Silverstein (U.S. Pat. No. 4,646,722) and Sidall (U.S. Pat. No. 4,741,326), whose disclosures are incorporated herein by reference. These methods attempt to prevent contamination of the endoscope, either by adding disposable working channels external to the endoscope itself (Silverstein) or by adding a disposable liner inside a working channel of the endoscope (Sidall).

Voloshin (U.S. Pat. No. 6,485,409), whose disclosure is incorporated herein by reference, describes an endoscope, which comprises an endoscopic probe, a bending section for directing the probe within the colon (steering unit), an insertion tube and a flexible covering sleeve or a sheath, which is coupled proximally to the probe. The sleeve is attached to the endoscope in such a manner that its folded section is retained between a cap and an internal spindle located between the insertion tube and the probe head. When inflated, the folded section unfolds over a flange of the internal spindle and an inner portion of the sleeve is pulled in a distal direction. The sleeve at the same time covers the insertion tube and prevents its contamination during the endoscopic procedure.

Eizenfeld (WO 2004/016299; PCT/IL03/000661), whose disclosure is incorporated herein by reference, discloses an endoscope, which employs a flexible inflatable sleeve assisting propulsion of the insertion tube within the body organ. The sleeve is retained in folded condition within a dedicated dispenser. The insertion tube is inserted into a dispenser and is advanced within the body organ. The insertion tube engages the sleeve, which covers the insertion tube and protects it from contamination.

Bar-Or (WO 2005/110185; PCT/IL05/000426), whose disclosure is incorporated herein by reference, discloses a disposable set, which comprises a dispenser for retaining a folded disposable sleeve, which upon inflation unfolds and protects the insertion tube from contamination.

The above-mentioned references teach how the principle of a disposable covering sleeve can be realized essentially in a colonoscopic apparatus, however they do not disclose how to implement this approach for preventing contamination in a gastroscopic apparatus as well.

This approach in connection with the gastroscopic apparatus is disclosed in our International patent application PCT/IL06/000279 herein incorporated by reference.

In this application is disclosed gastroscopic apparatus for inspecting a body channel. The apparatus comprises an insertion tube, an operating handle, a dispenser, which is detachably coupled to the insertion tube, a covering sleeve, which is retained within the dispenser, and a mouthpiece, which is provided with a means for arresting the dispenser within the mouthpiece. Upon arresting the dispenser and advancement of the insertion tube within the body channel, the covering sleeve extends from the dispenser and deploys around the insertion tube to protect from contamination that portion of the insertion tube, which had passed the dispenser.

The disadvantage of this gastroscopic apparatus is associated with its relative complicate design, which requires separate dispenser and mouthpiece. Accordingly manufacturing of the apparatus, its assembling and preparation to the endoscopic procedure becomes a complicate task, which would be desirable to simplify.

### SUMMARY OF THE INVENTION

The object of the present invention is to provide a new and improved gastroscopic apparatus, which minimizes the risk of cross contamination to both patient and staff during the gastroscopic procedure.

Still a further object of the invention is to provide a new and improved gastroscopic apparatus, wherein an insertion tube can be advanced along the esophagus while being protected by a disposable covering sleeve.

Still a further object of the invention is to provide a new and improved gastroscopic apparatus fitted with a dispenser for storing the covering sleeve in a compact state before insertion the insertion tube in the esophagus and before displacement thereof along the esophagus.

And yet another object of the invention is to provide a new and improved gastroscopic apparatus, which is provided with a simple design by virtue of combining the dispenser with a mouthpiece in an integral component suitable for storing the sleeve and at the same time suitable for holding by patient's teeth.

For a better understanding of the present invention as well of its benefits and advantages, reference will now be made to the following description of its embodiments, taken in combination with the accompanying drawings.

### BRIEF DESCRIPTION OF THE DRAWINGS

FIG. 1 is a perspective view of an operation handle of a gastroscopic apparatus.

FIG. 2 is a general view of the gastroscopic apparatus.

FIGS. 3-6 show how prior art gastroscopic apparatus provided with separate dispenser and mouthpiece is used.

FIG. 7 is an exploded isometric view of an integral component, functioning as combined dispenser and mouthpiece.

FIG. 8 is an isometric view of the integral component together with a fragment of the insertion tube.

FIG. 9 is an isometric view of the distal end of the integral component.

FIG. 10 is an isometric view of the proximal end of the integral component.

FIG. 11a is a side view of the outer housing portion of the integral component.

FIG. 11b is an enlarged view of detail A encircled in FIG. 11a.

FIG. 12a is a side view of the inner bushing portion of the integral component.

FIG. 12b is an enlarged view of detail B encircled in FIG. 12a.

FIG. 13a is a cross-sectional view of the outer housing portion with the bushing portion received therein but without the covering sleeve.

FIG. 13b is an enlarged view of detail C encircled in FIG. 13a.

FIG. 14 is a cross-sectional view of the integral component with a covering sleeve stored in the dispenser and with a cap put on the distal end of the insertion tube.

FIG. 15 is a side view of the integral component showing specific geometry of the bite portion designed for reliable holding by patient's teeth.

### DETAILED DESCRIPTION OF THE INVENTION

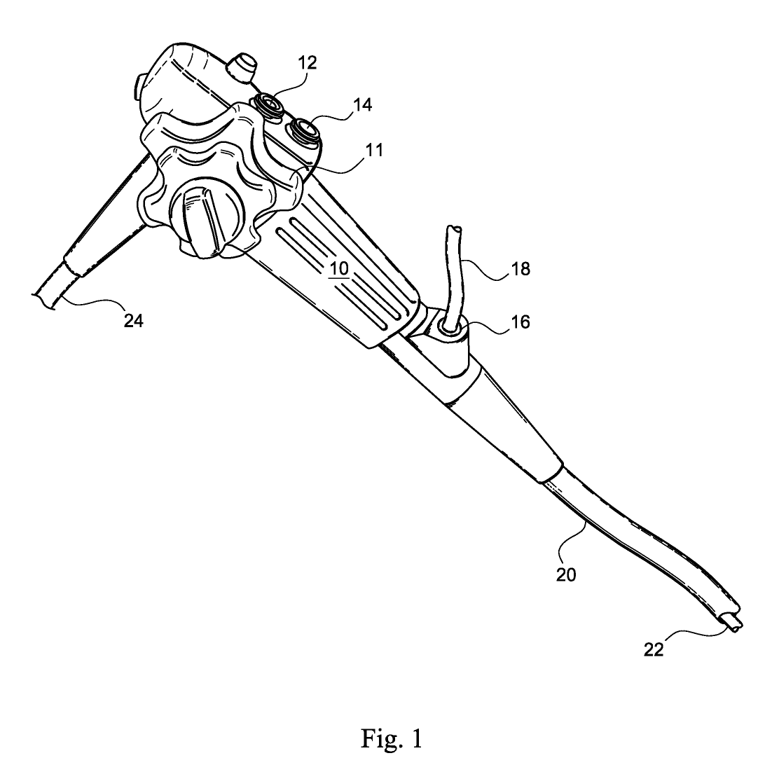

With reference to FIG. 1 an operation handle 10 of a gastroscopic apparatus of the present invention is shown. The operation handle is one of the major components of the apparatus. The operation handle employed in the apparatus of the present invention is of a conventional design and is identical with the operation handle of a prior art gastroscopic apparatus in all its functionality for the operator. Still further components of the gastroscopic are a system control unit (further referred-to as SCU) and a monitor. These components are also similar to those of the prior art gastroscopic apparatus, described in our International patent application PCT/IL06/000279 herein incorporated by reference.

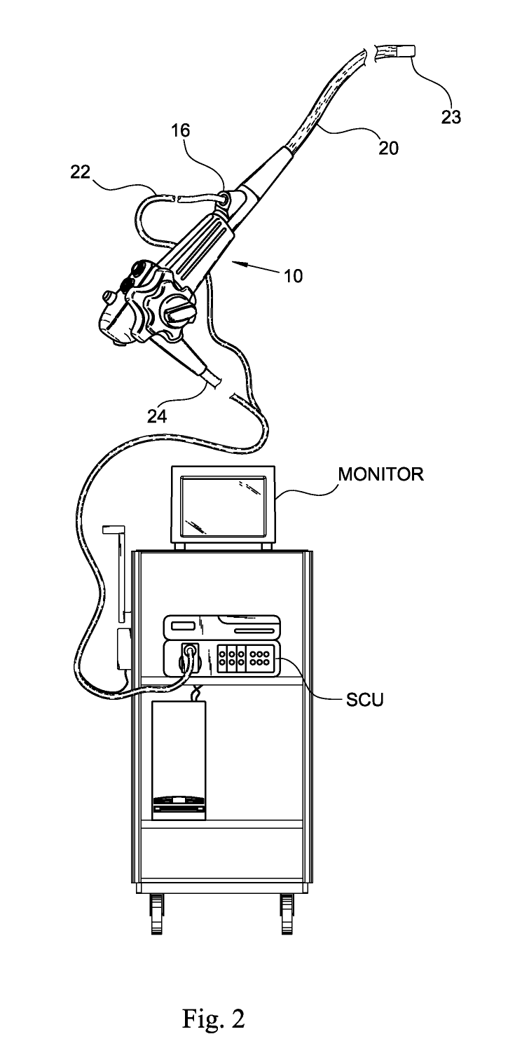

The system control unit and the monitor are not shown in FIG. 1, but are seen in FIG. 2.

The operation handle includes an angulation control knob or wheel 11, suction and air/water buttons 12, 14 for admitting fluid medium into the esophagus and a Y-connector 16 provided with an inlet port 18 and a channel for connecting to respective sources of fluid medium (usually air, water and vacuum). These sources are available at the SCU.

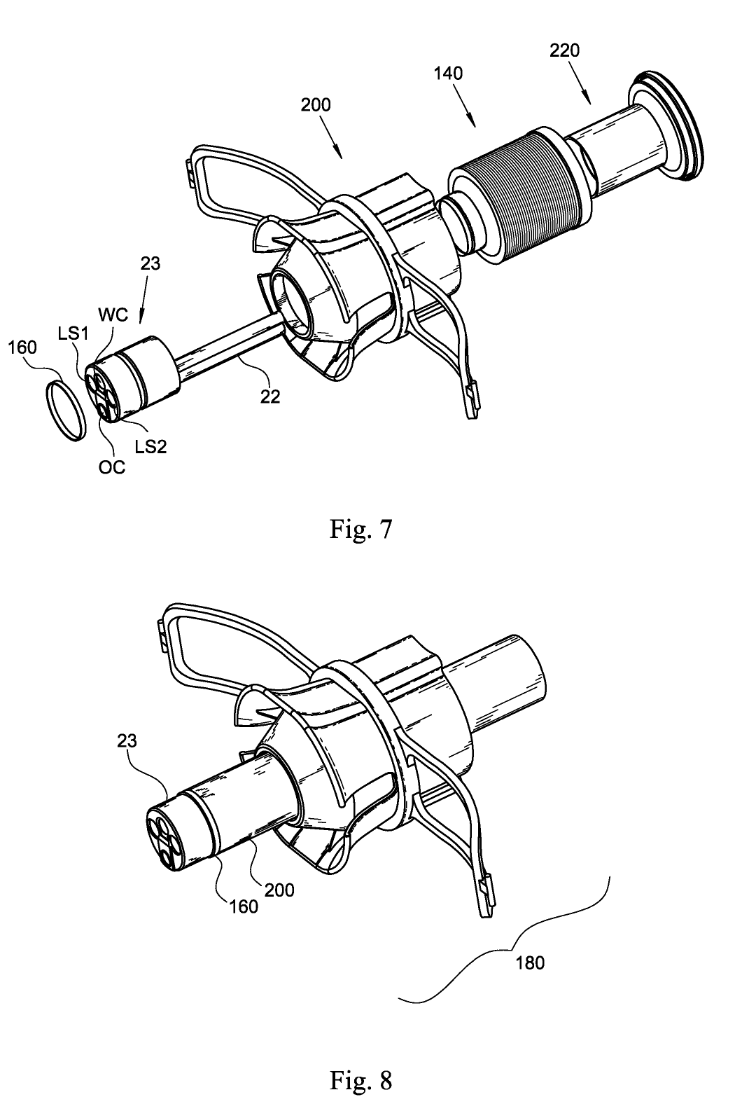

As seen in FIG. 2 the operation handle is operatively connected to a proximal end of an insertion tube 20, through which a guide channel 22 extends. A cap 23 is put on an optical camera head provided at the distal end of the insertion tube. The cap is defined by a forward butt end and by a cylindrical periphery surface. As will be shown further with reference to FIG. 7 the butt end is conveniently provided with windows, which are aligned with an optical camera located within the optical head and with a light source of the optical camera. In accordance with a preferred embodiment a CCD camera should be used. The optical head is fitted also with an integrated light source, preferably white LED light source. By virtue of the CCD camera and the LED light source, visualization is much more efficient since there is no need for fiber optics, and thus it is possible to significantly reduce maintenance and repair costs. An example of a suitable optical head provided with the CCD camera and the LED light source can be found in our patent application PCT/IL05/000929 whose disclosure is incorporated herein by reference.

In a preferred embodiment of the invention the guide channel extends along the insertion tube. The guide channel is designed as an integral conduit suitable for receiving a disposable tube with separate lumens for supplying air, water and vacuum. This tube will be referred-to further as multilumen tubing. An example of such multilumen tubing and its description can be found in Bar-Or (WO 2005/110200; PCT/IL05/000428), whose disclosure is incorporated herein by reference. The operation handle is connected also to a system control unit (SCU, shown in FIG. 2) via an umbilical cord 24, through which extend electrical cables connecting the SCU with the optical head deployed in the distal end of the insertion tube. All above elements are similar to those employed in the conventional gastroscopic apparatuses.

It is not shown in detail, but one should keep in mind that within the insertion tube are provided various devices, which are necessary for proper functioning of the endoscope. These devices and their arrangement within the insertion tube are known in the art. Among such devices one can mention vertebrae and strings, which can be manipulated by the angulation knob provided at the operation handle.

It should be also borne in mind that in the gastroscopic apparatus of the invention is employed a disposable sleeve which during the gastroscopic procedure feeds out from a dispenser provided at the distal end of the insertion tube and covers it. Therefore the dispenser is another important component of the apparatus. The dispenser arrangement in connection with the endoscopic apparatuses in general and gastroscopic apparatuses in particular is disclosed in our International patent applications PCT/IL03/000661, PCT/IL05/000426 and PCT/IL06/000279, whose disclosures are incorporated herein by reference.

Still further component of the gastroscopic apparatus is a mouthpiece, which the patient holds by teeth in his/her mouth during the gastroscopic procedure. The insertion tube is introduced into and evacuated from the patient's mouth through the mouthpiece. This arrangement is disclosed in our International patent application PCT/IL06/000279. According to this arrangement during the gastroscopic procedure the mouthpiece is held within the patient's mouth by her or his teeth and the insertion tube is advanced along the esophagus through the mouthpiece, while the dispenser is arrested within the mouthpiece.

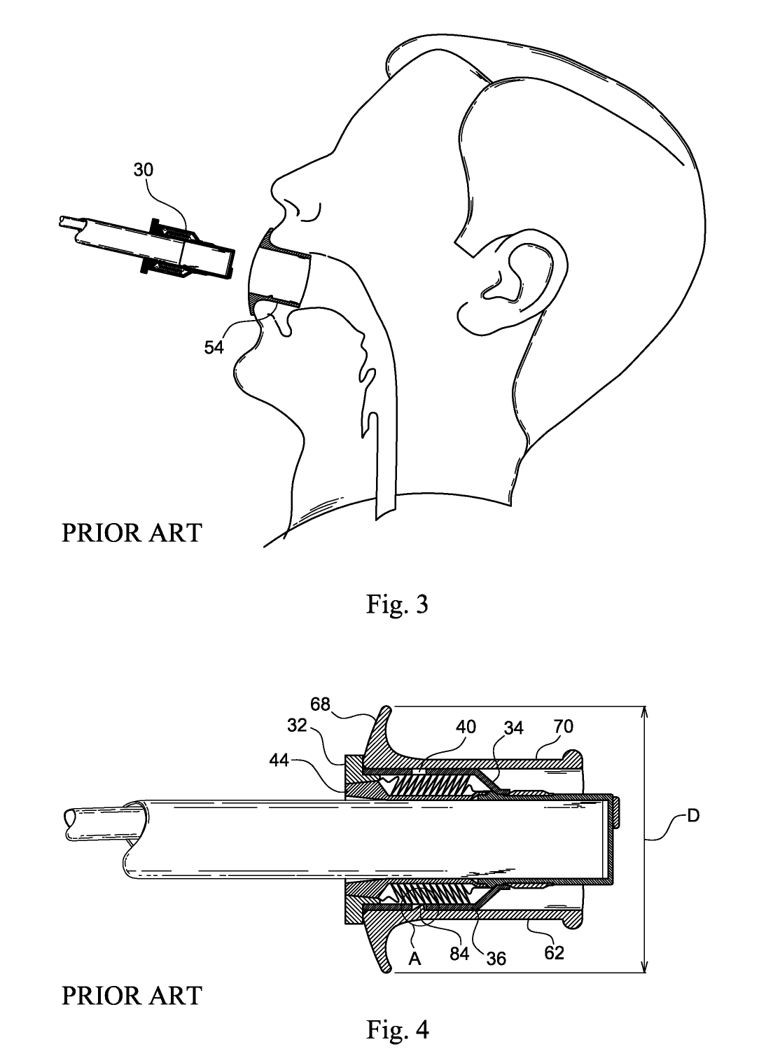

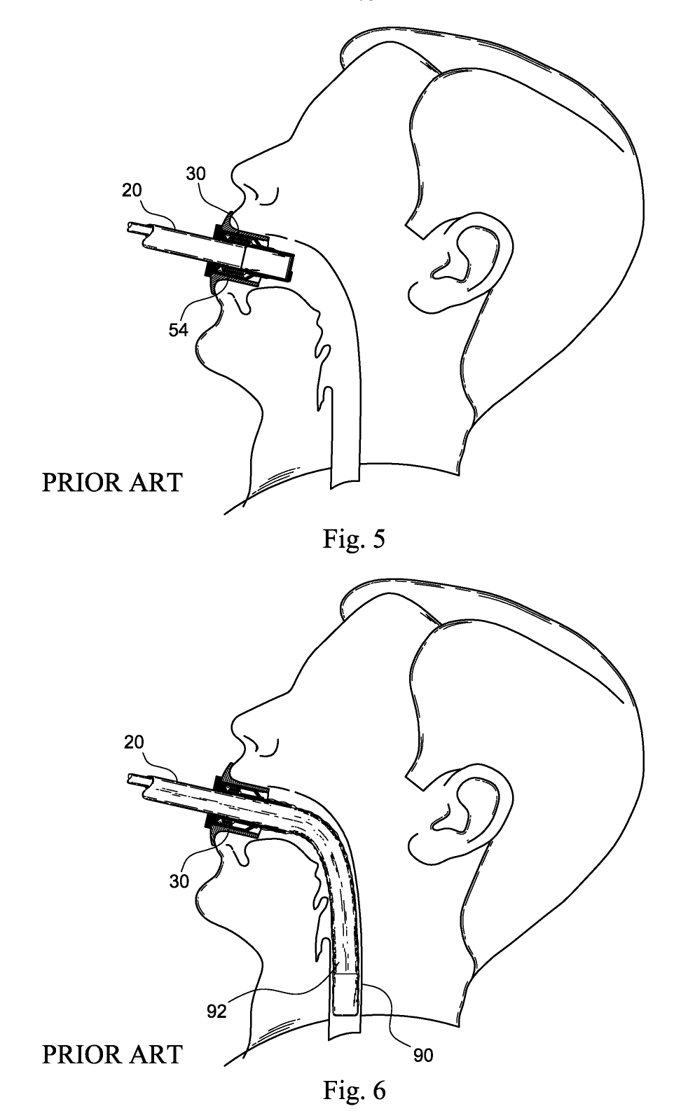

Referring now to FIG. 3-6 it is shown the principle of operation of a prior art gastroscopic apparatus, in which there is employed separate mouthpiece 54, held by the patient's teeth and separate dispenser 30, deployed at the distal end of the insertion tube. The dispenser and the mouthpiece constitute two separate components, which should be assembled before the gastroscopic procedure. Upon assembling the dispenser remains arrested in the mouthpiece during the endoscopic procedure. As seen in FIGS. 5, 6 the insertion tube is gradually protracted through the mouthpiece into esophagus and protection sleeve feeds out from the dispenser and covers the insertion tube.

The dispenser of the prior art gastroscopic apparatus comprises several parts, which should be assembled together for receiving the sleeve and after that the dispenser could be affixed to the mouthpiece. The gist of the present invention is to provide a single, integral component, which has very simple design and in which the dispenser is combined with the mouthpiece.

Such integral component from one side would render the manufacturing easier and cheaper and from the other side would render the preparation of the apparatus to the gastroscopic procedure more convenient and fast.

Referring now to FIG. 7 there is shown exploded view of an assembly comprising dispenser, mouthpiece, covering sleeve, multilumen tubing and a cap to be put on the optical head of the gastroscopic apparatus. The assembly or set is shown without the insertion tube. This situation corresponds to an initial stage of preparation the endoscopic apparatus provided with protective covering sleeve. More detailed description of this preparation procedure can be found in our International patent application PCT/IL05/000426 whose specification is incorporated herein by reference. It is seen that the assembly comprises an outer housing portion 200, an inner bushing portion 220, a covering sleeve 140, which is folded as a concertina, a cap 23 wearable on an optical head provided at a distal end of the insertion tube (not seen) and attached to a multilumen tubing 22 protruding proximally from the cap. A snap ring 160 for anchoring distal end of the sleeve at the cap is also seen. Provided in the butt end of the cap are seen windows LS1, LS2 for the light sources, window OC for the optical camera are seen as well as opening WC for the working channel of the multilumen tubing.

FIG. 8 shows the assembly at a final stage of the preparation procedure when the covering sleeve is deployed within the combined dispenser and mouthpiece, the multilumen tubing is inserted within a working channel of the insertion tube and the cap is put on the optical head. At this stage the inner bushing portion is received within the outer housing portion and insertion tube 20 is seen protruding distally from the outer housing portion being ready for insertion within the patient's mouth. The bushing portion is reliably secured within the outer housing portion for example by virtue of a snap arrangement, which will be explained further.

The combined dispenser and mouthpiece is shown in FIG. 8 as an integral component designated by common reference numeral 180. This integral component is manufactured from suitable plastic material, e.g. polyamide, polyethylene or polypropylene. The required geometry of the component, which will be explained further, is obtained by suitable forming process, e.g. injection molding.

Referring to FIG. 8 it is seen also that snap ring 160 is put on the cap and secures on it a portion of the covering sleeve. This portion now covers the remainder of the cap and the insertion tube behind the cap.

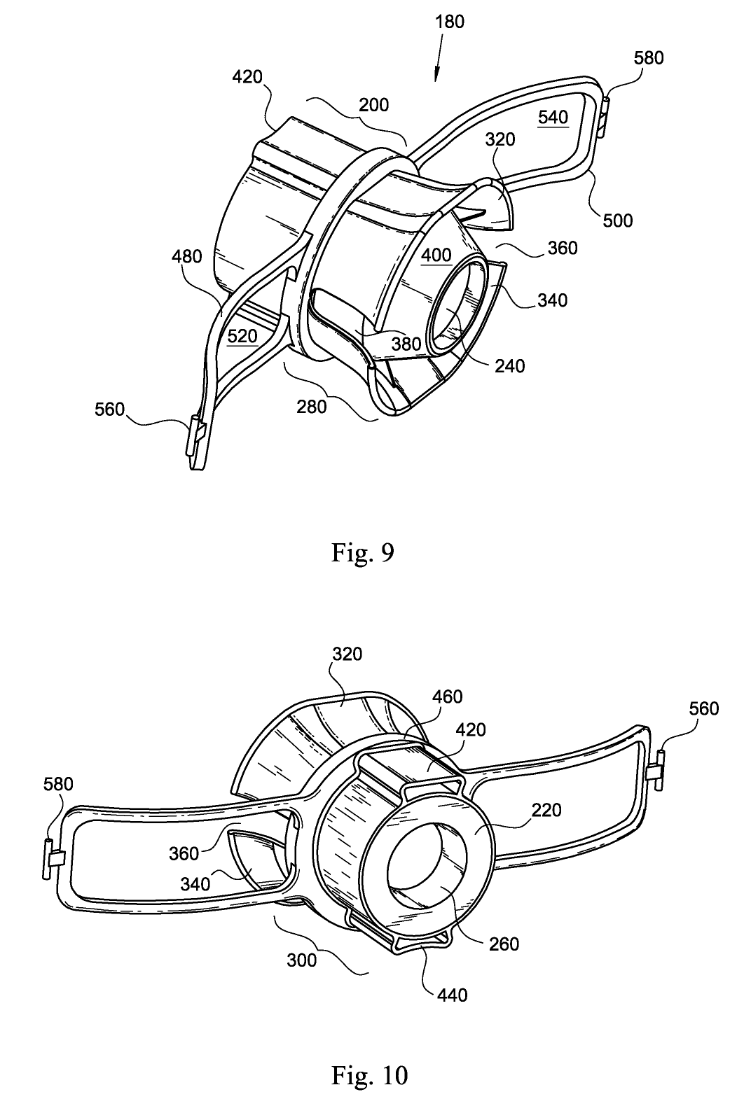

Referring now to FIGS. 9-10 the integral component 180 will be explained in more details. The integral component comprises outer housing portion 200 and inner bushing portion 220, which is receivable in the housing portion. A longitudinal passage goes through the integral component from a distal opening 240 provided in the housing portion to a proximal opening 260, provided in the bushing portion. Through this passage the insertion tube can be displaced either distally or proximally during the gastroscopic procedure.

The outer housing portion comprises a forward, bite portion 280 and a rear, dispenser portion 300. The bite portion is provided with two flaring skirt regions 320, 340, which are configured to be conveniently and reliably held by the patient's teeth during the gastroscopic procedure. The skirt regions are configured in such a manner that their curvature allows reliable holding of the bite portion by the patient's teeth. This will be explained further with reference to FIG. 15.

Two gap regions 360, 380 divide between the skirt regions. Surrounded by the skirt regions a conically narrowing dome portion 400 is provided in the forward mouthpiece portion. The distal opening 240 is made in the dome portion.

The dispenser portion comprises also two oppositely situated ramps 420, 440 which protrude from the dispenser portion. The doctor's fingers are abutted by the ramps during the gastroscopic procedure. It is advantageous if abutting surface of at least one of the ramps is curved to enable more convenient contact with the doctor's fingers. In FIG. 10 the curved ramp is designated by reference numeral 440.

The bite portion and the dispenser portion are connected to an intermediate rim portion 460, provided with two lateral wings 480, 500. The wings are situated diametrically at both sides of the rim portion. To reduce weight of the integral component, respective openings 520, 540 are provided within the wings. The openings 520,540 and the gap regions 360,380 allow convenient access to the patient's mouth by the doctor's finger when the doctor needs to exert pressure on the patient's tongue. This some times might be desired at the beginning of the gastroscopic procedure in order to direct the insertion tube properly into esophagus.

It is seen also that each wing is provided with a respective cleat 560, 580 for fastening respective ends of an elastic strap (not shown). During gastroscopic procedure the strap is worn behind the back of patient's head to hold the mouthpiece more reliably in place within the patient's mouth. This arrangement is disclosed in our International patent application PCT/IL06/000279.

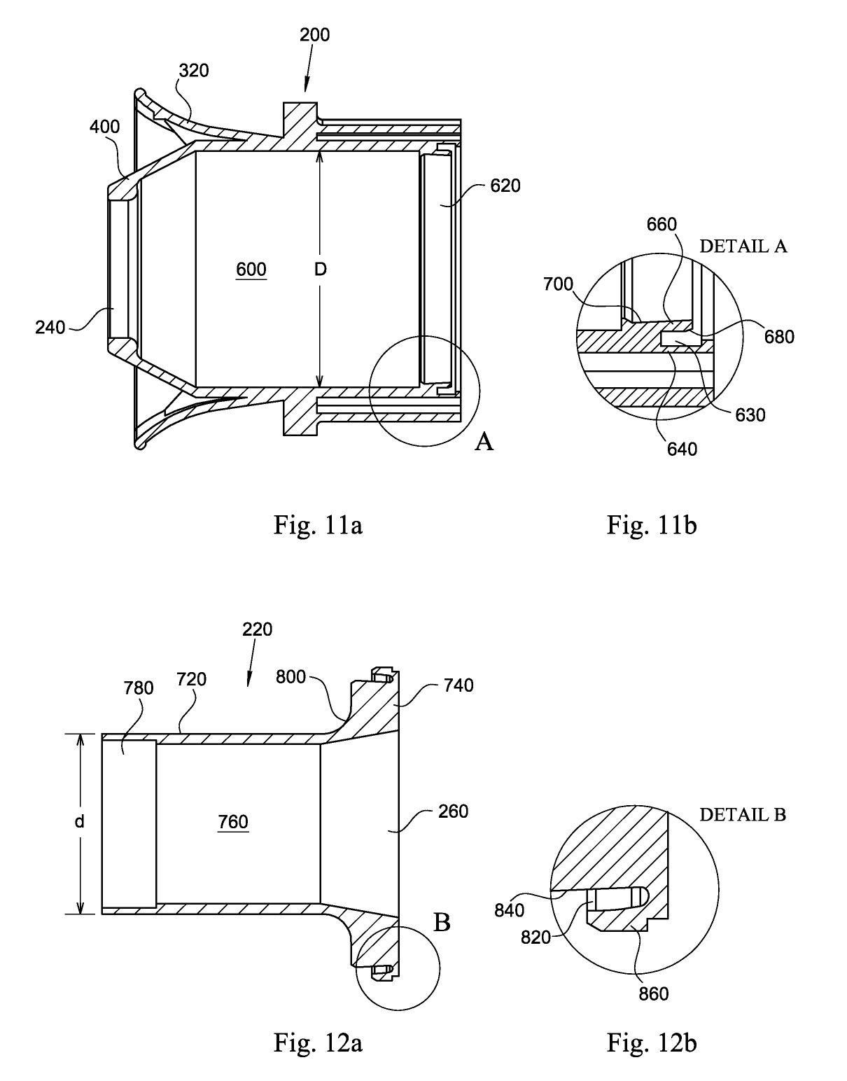

Referring now to FIGS. 11a, 11b, 12a and 12b the outer housing portion and the inner bushing portion will be explained. The already mentioned elements are designated in these figures by the same reference numerals as in FIGS. 9, 10.

As seen in FIG. 11a the outer housing portion is configured as a tubular body having an inner cylindrical passage 600 extending between a proximal opening 620 and distal opening 240. Diameter of the cylindrical passage is D. Referring now to FIG. 11b it is seen that the proximal opening is configured with an annular recess 630 defined by a long wall 640 and a short wall 660 having deflecting section 680. The inwardly facing surface 700 of the short wall is inclined with respect to the longitudinal axis of the housing portion and provides a first conical surface.

With reference to FIG. 12a it is seen that the inner bushing portion is configured as a tubular body having cylindrical main body portion 720 and a rear flange portion 740. The outside diameter of the main body portion is d and it is less than diameter D of the cylindrical passage 600. By virtue of this provision when the bushing portion is received in the housing portion there is provided an annular space therebetween and covering sleeve 140 is stored in this space.

An inner through going cylindrical passage 760 extends along the bushing portion from proximal opening 260 to a distal opening 780. Through this passage the insertion tube can be displaced proximally or distally during the gastroscopic procedure.

The flange portion smoothly transforms into the main body portion through a narrowing rounded region 800.

Referring to FIG. 12b it is shown that flange portion is provided with an annular recess 820, which is defined between an outwardly facing surface 840 and an external wall 860.

Outwardly facing surface 840 is inclined with respect to the longitudinal axis of the housing portion and provides a second conical surface. The inclination of the first conical surface 700 and the second conical surface 840 is about 2 degrees, such that when the bushing portion is received in the housing portion there is provided frictional engagement between the conical surfaces. This engagement is used for anchoring the proximal end of the covering sleeve in the dispenser.

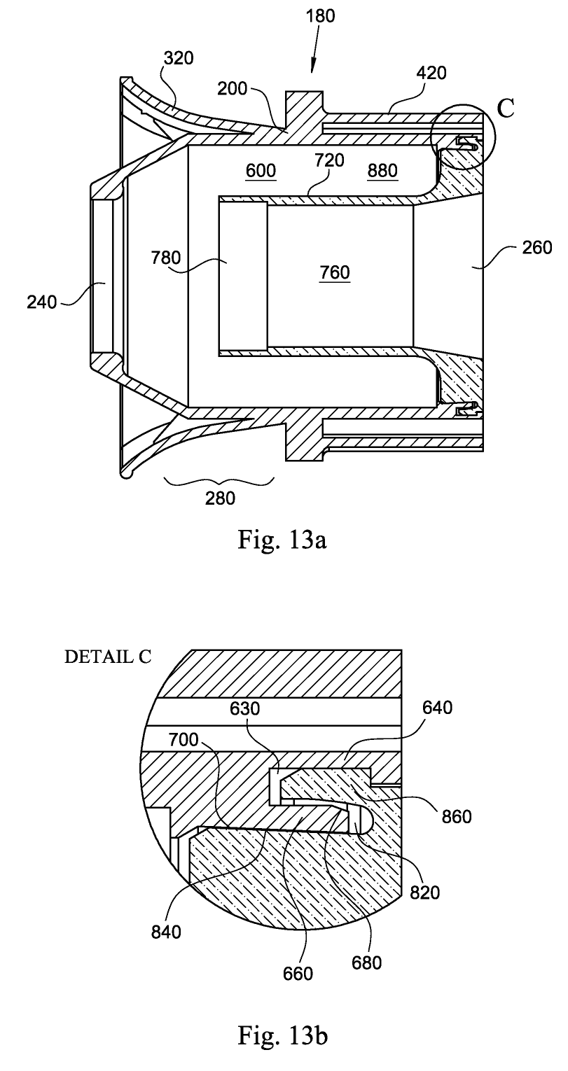

When the bushing portion is being inserted within the dispenser portion, as shown in FIG. 13a, 13b external wall 860 constitutes a male component, deformable by deflecting section 680. The male component is receivable in recess 630, which constitutes a female component. Thicknesses of short wall 660, of external wall 640 as well as direction of deflecting section 680 are selected in such a manner that upon entering the bushing portion into dispenser portion there is provided elastic snapping engagement therebetween. By virtue of this snapping engagement the flange portion is securely arrested in the proximal opening of the dispenser portion.

In FIG. 13a, 13b is shown integral component 180 in an assembled state when the bushing portion is arrested in the outer housing portion. As mentioned above since the outside diameter d of the main body portion is less than diameter D of the passage 600 an annular space 880 is provided therebetween. This space is used for storing the covering sleeve in the combined component, which therefore constitutes a dispenser. At the same time by virtue of bite portion 280 the combined component functions also as a mouthpiece.

In FIG. 13b the flange portion is shown after it has been arrested in the housing portion. Conical surfaces 700, 840 are in frictional engagement by virtue of small inclination angle.

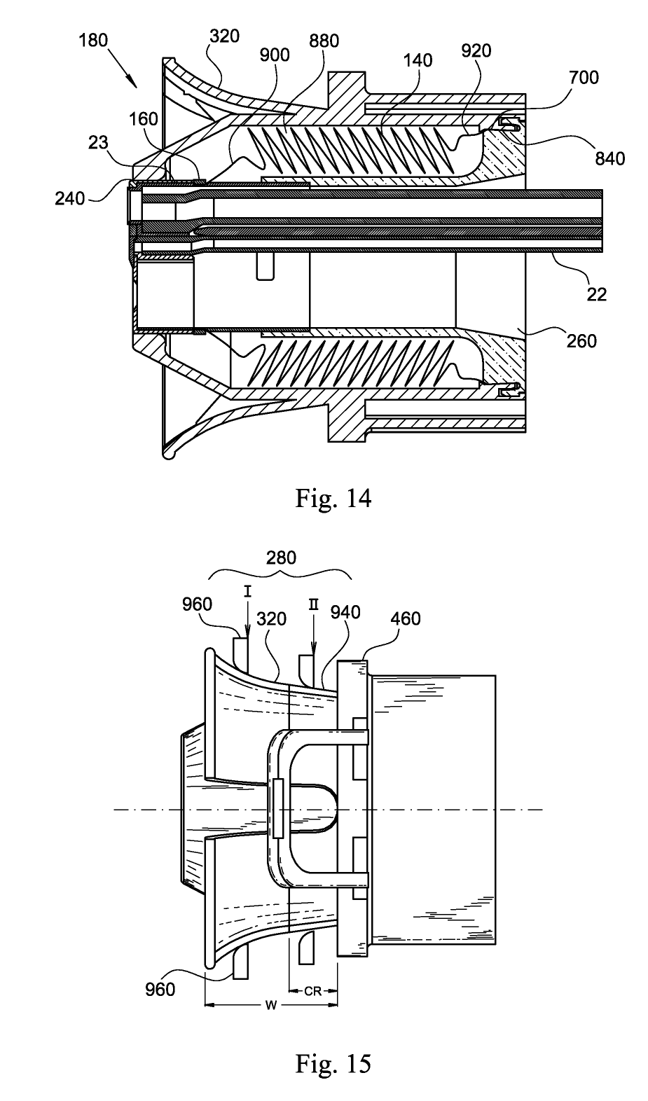

In FIG. 14 the integral component is shown when it stores folded covering sleeve 140 within annular space 880. Distal end 900 of the sleeve is anchored between ring 160 and outwardly facing surface of cap 23. Proximal end 920 of the sleeve is anchored between conical surface 840 of the flange portion and conical surface 700 of the housing portion. Multilumen tubing 22 extends along the combined component and is ready for engagement with the insertion tube whereupon the apparatus can be used in the gastroscopic procedure. During the gastroscopic procedure the mouthpiece portion of the integral component is held within the patient's mouth so that the insertion tube can be advanced into patient. Proximal end of the covering sleeve is secured at the flange portion. When the insertion tube is pushed distally through the combined component it urges the sleeve's distal end to move distally together with the insertion tube. Distal end of the covering sleeve extends from the dispenser portion of the combined component, unfolds and covers a portion of the insertion tube, which has passed through the dispenser portion. It can be appreciated that the unfolded sleeve deploys around the insertion tube and reliably protects it from any contamination matter originating from the esophagus.

Referring now to FIG. 15 it is shown how specific geometry of bite portion 280 assists for reliable holding thereof in the patient's mouth. It is seen that the bite portion is provided with skirt region 320, which comprises a conical region 940 and a rounded region. The conical region begins at the rim portion 460 and smoothly transfers into rounded region. The rounded region widens outside. The geometry of the bite portion is configured in such a manner that when patient's teeth 960 bite the skirt region they gradually slide from an initial position towards the conical region and then along the conical region to a final position adjacent the intermediate rim portion. The initial position of the teeth is designated I. The final position of the teeth is designated II. One can readily appreciate that by virtue of this provision the bite portion is always remained within the mouth being reliably held between the teeth. In practice to obtain such a geometry the conical region should have inclination of about 8-10 degrees, the rounded portion should have radius of about 13-15 mm and the length CR of the conical region should be about 0.5 of width W of the bite portion.

1. an endoscopic apparatus for inspecting a body channel of gastrointestinal: system of a patient, said apparatus comprising
an insertion tube provided at a distal end thereof with an optical head for visualization the body channel,
an operating handle with a means for navigating the distal end of the insertion tube during advancement thereof along the body channel and with a means for controlling the supply of a fluid medium to the body channel,
a covering sleeve deployable on the insertion tube,
a combined component, comprising an outer housing portion and an inner bushing portion, said inner bushing portion being receivable within the housing portion such that an annular space is provided therebetween for retaining the covering sleeve thereinto, and said outer housing portion is provided with a bite portion, to be grasped between the patient's teeth, and said bushing portion is provided with a longitudinal passage through which said insertion tube can be either protracted or retracted during inspecting the body channel.

2. The endoscopic apparatus as defined in claim 1, in which the outer housing portion comprises a forward mouthpiece portion and a rear dispenser portion.

3. The endoscopic apparatus as defined in claim 1, in which said bite portion comprises at least two skirt regions, which are configured and dimensioned to enable convenient and reliable holding by the patient's teeth.

4. The endoscopic apparatus as defined in claim 3, in which said skirt regions are separated by gaps.

5. The endoscopic apparatus as defined in claim 1, in which said passage is provided with a distal opening made in the mouthpiece portion and a proximal opening made in the inner bushing portion.

6. The endoscopic apparatus as defined in claim 1, in which said rear dispenser portion is provided with at least one abutting ramp for abutting the doctor's fingers during inspecting the body channel.

7. The endoscopic apparatus as defined in claim 3, in which said forward mouthpiece portion and said rear dispenser portion are connected to an intermediate rim portion.

8. The endoscopic apparatus as defined in claim 7, in which said rim portion is provided with two lateral wings.

9. The endoscopic apparatus as defined in claim 1, in which the proximal opening is configured with a first annular recess made in the housing portion, said first annular recess is defined by a long wall and a short wall, such that an inwardly facing surface of the short wall is inclined with respect to a longitudinal axis of the housing portion and provides a first conical surface, whereas the proximal opening is configured also with a second annular recess made in the bushing portion, said second annular recess is defined by an outwardly facing surface and an external wall, said outwardly external wall is inclined with respect to the longitudinal axis of the housing portion and provides a second conical surface, the arrangement being such that upon receiving the bushing portion within the housing portion a frictional engagement is provided between the first and the second conical surface.

10. The endoscopic apparatus as defined in claim 9, in which a proximal end of said covering sleeve is anchored between the first conical surface and the second conical surface.

11. The endoscopic apparatus as defined in claim 9, in which said bushing portion is provided with a flange portion, which is snappingly engageable within the first annular recess made in the said dispenser portion such that said inner bite portion is arrestable in the said outer housing portion.

12. The endoscopic apparatus as defined in claim 7, in which said at least one of the said skirt regions is configured with a conical region and with a rounded region, such that when the patient's teeth bite the skirt region they gradually slide from an initial position towards the conical region and then along the conical region to a second final position adjacent the rim portion.

13. The endoscopic apparatus as defined in claim 1, in which said apparatus is a gastroscope.

14. A combined component for use with an endoscopic apparatus for inspecting a body channel of gastrointestinal system of a patient, said apparatus comprising an insertion tube coverable by a covering sleeve deployable on the insertion tube upon advancing the insertion tube into the body channel, said combined component comprising an outer housing portion and an inner bushing portion, said inner bushing portion being receivable within the housing portion such that an annular space is provided therebetween for retaining the covering sleeve thereinto, and said outer housing portion is provided with a bite portion, to be grasped between the patient's teeth, and said bushing portion is provided with a longitudinal passage through which said insertion tube can be either protracted or retracted during inspecting the body channel.

15. The component as defined in claim 14, in which the outer housing portion comprises a forward mouthpiece portion and a rear dispenser portion.

16. The component as defined in claim 14, in which said bite portion comprises at least two skirt regions, which are configured and dimensioned to enable convenient and reliable holding by the patient's teeth.

17. The component as defined in claim 16, in which said skirt regions are separated by gaps.

18. The component as defined in claim 14, in which said passage is provided with a distal opening made in the mouthpiece portion and a proximal opening made in the inner bushing portion.

19. The component as defined in claim 15, in which said forward mouthpiece portion and said rear dispenser portion are connected to an intermediate rim portion.

20. The component as defined in claim 14, in which the proximal opening is configured with a first annular recess made in the housing portion, said first annular recess is defined by a long wall and a short wall, such that an inwardly facing surface of the short wall is inclined with respect to a longitudinal axis of the housing portion and provides a first conical surface, whereas the proximal opening is configured also with a second annular recess made in the bushing portion, said second annular recess is defined by an outwardly facing surface and an external wall, said outwardly external wall is inclined with respect to the longitudinal axis of the housing portion and provides a second conical surface, the arrangement being such that upon receiving the bushing portion within the housing portion a frictional engagement is provided between the first and the second conical surface.

21. The component as defined in claim 20, in which a proximal end of said covering sleeve is anchored between the first conical surface and the second conical surface.

22. The component as defined in claim 20, in which said bushing portion is provided with a flange portion, which is snappingly engageable within the first annular recess made in the said dispenser portion such that said inner bite portion is arrestable in the said outer housing portion.
